(19) United States
(12) Patent Application Publication
(10) Pub. No.: US 2015/0111281 A1
(43) Pub. Date: Apr. 23, 2015

(54) Production of Porphyrin-Containing Polypeptides in the Presence of Formate

(71) Applicant: Danisco US Inc., Palo Alto, CA (US)

(72) Inventor: Evan Sims, San Francisco, CA (US)

(73) Assignee: DANISCO US INC., Palo Alto, CA (US)

(21) Appl. No.: 14/360,918

(22) PCT Filed: Nov. 20, 2012
(86) PCT No.: PCT/US2012/066106

§ 371 Date: May 27, 2014

Related U.S. Application Data
(60) Provisional application No. 61/564,232, filed on Nov. 28, 2011.

(51) Int. Cl. C12N 9/08

(52) U.S. Cl. CPC C12N 9/0065; C12Y 111/01006
USPC 435/192

(57) ABSTRACT

The present compositions and methods relate to the production of polypeptides in submerged culture, and specifically the large-scale production of porphyrin-containing polypeptides, e.g. catalase, by microbial fermentation in the presence of formate.

### PRIORITY

The present application claims priority to U.S. Provisional Patent Application Ser. No. 61/564,232, filed on Nov. 28, 2011, which is hereby incorporated by reference in its entirety.

### TECHNICAL FIELD

The present compositions and methods relate to the production of polypeptides in submerged culture, and specifically to the large-scale production of porphyrin-containing polypeptides by microbial fermentation.

### BACKGROUND

Microbially-expressed polypeptides are used in a number of industrial and domestic applications, including textile processing, fabric care, ethanol production, brewing, and baking, to name a few. Polypeptides are typically provided to end users in stabilized dry or liquid formulations to maintain their activity.

Some microbially-expressed polypeptides contain porphyrins, i.e., heterocyclic macrocycles composed of four interconnected modified pyrrole subunits coordinated with a metal atom, such as iron, magnesium, copper, nickel, cobalt, molybdynum, and vanadium. Porphyrin-containing polypeptides can be stabilized with formate, which is conventionally added to the polypeptides as a formulation ingredient.

### SUMMARY

The present compositions and methods relate to the production of porphyrin-containing polypeptides in fungal cells grown in submerged culture in the presence of formate.

In one aspect, a method for producing a porphyrin-containing polypeptide in host cells is provided, comprising: growing host cells that produce a porphyrin-containing polypeptide in a suitable culture medium to produce a fermentation broth; adding to the fermentation broth an effective amount of formate; and recovering the porphyrin-containing polypeptide produced; wherein the presence of formate in the fermentation broth increases the activity, stability, or expression levels of the porphyrin-containing polypeptide, compared to growing the same host cells in the equivalent culture medium in the absence of formate.

In some embodiments, the host cells are grown to fed-batch stage prior to adding the formate. In some embodiments, the growth of the host cells is substantially complete prior to adding the formate. In some embodiments, the host cells are grown to a stage of being ready to induce for production of the porphyrin-containing polypeptide prior to adding the formate. In some embodiments, the culture medium comprises glucose, and the glucose is substantially depleted by the host cells prior to adding the formate.

In some embodiments, the concentration of formate in the fermentation broth is less than or equal to about 2.0 grams formate per kilogram fermentation broth. In some embodiments, the cumulative amount of formate added during fermentation is from about 0.007 to about 1.35 mole formate per kilogram (kg) fermentation broth. In some embodiments, the amount of formate added is from about 0.03 to about 7.1 millimole formate per hour elapsed fermentation time per kilogram of fermentation broth. In some embodiments, the amount of formate added is from about 40 to about 4400 millimole formate per hour elapsed fermentation time per kilogram dry solids of substrate. In some embodiments, the amount of formate added is from about 50 to about 7500 mole formate per mole of active porphyrin-containing polypeptide.

In some embodiments, the formate is in the form of formic acid. In some embodiments, the formate is in the form of a formate salt. In some embodiments, the formate is in the form of sodium formate.

In some embodiments, the host cells are fungal cells. In some embodiments, the host cells are filamentous fungal cells. In some embodiments, the host cells are Trichoderma cells.

In some embodiments, the porphyrin-containing polypeptide is a heme-containing polypeptide. In some embodiments, the porphyrin-containing polypeptide is a catalase enzyme.

In another aspect, a porphyrin-containing polypeptide produced by any of the described methods is provided. In some embodiments, the porphyrin-containing polypeptide is a catalase enzyme.

These and other aspects and embodiments of the compositions and methods will be apparent from the present description and drawings.

### BRIEF DESCRIPTION OF THE DRAWINGS

FIG. 1 is a graph showing the amount of catalase activity present in cell broth over time in cell cultures grown in the presence or absence of formic acid or sodium formate.

FIG. 2 is a graph showing the amount of total amount of protein produced in cell broth over time in cell cultures grown in the presence or absence of formic acid or sodium formate.

FIG. 3 is a graph showing the amount of catalase activity present in cell broth, normalized for total protein production, over time in cell cultures grown in the presence or absence of formic acid or sodium formate.

### DETAILED DESCRIPTION

The present compositions and methods relate to the production of porphyrin-containing polypeptides in fungal cells grown in submerged culture in the presence of formic acid, or a salt of formic acid (herein, collectively referred to as “formate”). Formate is a known stabilizing agent for porphyrin-containing polypeptides that is conventionally added to recovered polypeptides following separation from cells and debris. While some bacteria can grow in the presence of formate, formate is a well-known fungicide, and is not conventionally added to submerged cultures of fungal cells.

It has surprisingly been found that formate can be added to fermentation broth (i.e., culture medium with cells) at the fed-batch stage of fungal fermentation, after most or all of the cell growth has taken place. The fed-batch stage of fermentation refers to time when the cells have substantially exhausted some of nutrients in the initial culture medium (e.g., glucose), and must be fed additional nutrients to maintain their metabolism. At this time, the cells are typically induced to produce a protein of interest, such as by the feeding of an inducing agent [e.g., 2-O-β-glucopyranosyl-D-glucose (sophorose), isopropyl-β-D-thio-galactoside (IPTG), and the like], depending on the particular organism and gene construction. The presence of formate at this stage in fermentation does not appear to affect cell viability, as evidenced by the rate of protein production. However, the presence of formate during fermentation does increase the yield of active porphyrin-containing polypeptides, presumably by stabilizing the polypeptides as they are produced. In this manner, porphyrin-containing polypeptides can be produced at lower cost and at higher titers.

The present compositions and methods are contemplated for use with a variety of different porphyrin-containing polypeptides. Exemplary polypeptides are endogenous or exogenous proteins of interest, particularly enzymes or binding proteins. Exemplary porphyrin-containing polypeptides include, but are not limited to, hemoglobins, myoglobins, oxidases, peroxidases, oxygenases, hemocyanins, chlorophylls, cytochromes, cobalamin, organophosphorus hydrolases, and catalases. Such enzymes typically contain porphyrin-metal complexes, most commonly based on iron, magnesium, and copper, although nickel, zinc, cobalt, molybdynum, and vanadium can be coordinated. Some polypeptides contain porphyrin-based prosthetic groups that are associated with particular coordinated metals, for example: heme (iron), chlorophyll (magnesium), hemocyanin (copper), and cobalamin (cobalt). A particular porphyrin-containing polypeptide, used to illustrate the present compositions and methods, is a catalase enzyme derived from Aspergillis niger. Catalase is a heme-containing polypeptide with a coordinated iron.

The compositions and methods are also contemplated for use with a variety of different host cells and fermentation media. In some embodiments, the host cells are fungal cells, including yeast and filamentous fungus cells. Examples of yeast include but are not limited to, Saccharomyces spp., Schizosaccharomyces spp., Arxula spp., Candida spp., Hansenula spp., Kluyveromyces spp., Pichia spp., or a Yarrowia spp. Examples of filamentous fungi include, but are not limited to, Trichoderma spp., Aspergillus spp., Fusarium spp., Scedosporium spp., Penicillium spp., Chrysosporium spp., Cephalosporium spp., Talaromyces spp., Geosmithia spp., Myceliophthora spp., and Neurospora spp. Particular filamentous fungus cells include, but are not limited to, Trichoderma reesei (previously classified as Trichoderma longibrachiatum and Hypocrea jecorina), Aspergillus niger, Aspergillus fumigatus, Aspergillus itaconicus, Aspergillus oryzae, Aspergillus nidulans, Aspergillus terreus, Aspergillus sojae, Aspergillus japonicus, Scedosporium prolificans, Neurospora crassa, Penicillium funiculosum, Penicillium chrysogenum, Talaromyces (Geosmithia) emersonii, Fusarium venenatum, Myceliophthora thermophila, and Chrysosporium lucknowense.

In other embodiments, the host cell is a bacterial cell, not previously known to grow in the presence of formate. Examples of suitable bacterial cells include but are not limited to, Gram-positive bacteria such as Bacillus subtilis, Bacillus licheniformis, Bacillus lentus, Bacillus brevis, Geobacillus stearothermophilus, Bacillus alkalophilus, Bacillus amyloliquefaciens, Bacillus coagulans, Bacillus circulans, Bacillus lautus, Bacillus megaterium, Bacillus thuringiensis, or Streptomyces lividans or Streptomyces murinus, or gram-negative bacteria such as E. coli. In yet further embodiments, the host cells are plant cells, such as algae, or animal cells, such as insect or mammalian cells.

Preferably the host cells are grown (i.e., “fermented”) in submerged liquid culture in a vessel or bioreactor. The culture can be of the batch or continuous variety, and may be aerobic or anaerobic. The porphyrin-containing polypeptides may be expressed as intracellular polypeptides, or as secreted polypeptides (i.e., using suitable signal sequence) to simplify recovery. Exemplary heterologous signal sequences are from B. licheniformis amylase (LAT), from B. subtilis (AmyE or AprE), and from Streptomyces Ce1A.

The particular cell medium depends on the host cells selected. Medium for the growth of fungi is well-known in the art. The pH of the medium may have to be adjusted following the addition of formate to cell broth. Formate is preferably introduced when the fungal cells growth is complete, or substantially complete (i.e., at least 80%, at least 85%, at least 90%, or even at least 95% complete, based on cell mass) and the cells are ready to induce to produce desired porphyrin-containing polypeptides. In some cases, formate is introduced when the initial amount of glucose present in the culture medium is substantially depleted (i.e., at least 80%, at least 85%, at least 90%, or even at least 95% depleted). Formate may be fed continuously or discontinuously into the fermentation broth to maintain a preselected target level of formate. However, maintaining an exact preselected level of formate is believed to be unnecessary.

The amount of formate added to the cell broth during fermentation (i.e., “an effective amount”), is selected to maximize the yield of active porphyrin-containing polypeptides, while avoiding levels of formate that significantly reduce cell viability, which can be measured based on protein expression, oxygen or nutrient consumption, pH, and the like. Without being limited to a theory, formate is believed to bind oxidized forms of porphyrin-containing polypeptides and reduce them to a stable form. However, formate is also degraded and metabolized by the cells, so the levels of formate in the fermentation broth will vary. In some cases, the amount of formate in the cell broth may become virtually undetectable.

Generally, the amount of formate maintained during fermentation, is up to (i.e., less than or equal to) about 2.0 grams (g) formate per kilogram (kg) fermentation broth, and preferably up to about 0.5 g formate per kg broth. Exemplary amounts are undetectable, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0 g formate per kg broth.

The cumulative amount of formate added during fermentation, may be expressed as from about 0.007 to about 1.35 mole formate per kilogram (kg) fermentation broth, and preferably from about 0.033 to about 0.41 mole formate per kg broth. Exemplary amounts are about 0.01, 0.03, 0.05, 0.07, 0.1, 0.2, 0.3, 0.4, 0.5, 0.6, 0.7, 0.8, 0.9, 1.0, 1.1, 1.2, and 1.3 mole formate per kg broth.

The amount of formate used can also be expressed as millimole (mmol) formate per hour elapsed fermentation time per kilogram of fermentation broth (mmol formate/hr/kg), and is generally from about 0.03 to about 7.1 mmol formate/hr/kg, and preferably from about 0.20 to about 2.1 mmol formate/hr/kg. Exemplary amounts are about 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, and 2.1 mmol formate/hr/kg.

The amount of formate used can also be expressed as millimole formate per hour elapsed fermentation time per kilogram dry solids of substrate (e.g., glucose, glycerol, etc.) added to the fermentation vessel (mmol formate/hr/kg DS fed), and is generally from about 40 to about 4400 mmol formate/hr/kg DS fed, and preferably from about 172 to about 1320 mmol formate/hr/kg DS fed. Exemplary amounts are about 200, 300, 400, 500, 600, 700, 800, 900, 1000, 1100, and 1200 mmol formate/hr/kg DS fed.

The amount of formate used can also be expressed as mole formate per mole of active porphyrin-containing polypeptide (mole formate/mole polypeptide), and is generally from about 50 to about 7500 mole formate/mole polypeptide, and preferably from about 250 to about 2260 mole formate/mole polypeptide. Exemplary amounts are about 300, 500, 700, 900, 1100, 1300, 1500, 1700, 1900, and 2100 mole formate/mole polypeptide.

Formate may be provided in the form of formic acid or a salt of formate, such as a sodium or potassium salt. In some embodiments, following recovery of the porphyrin-containing polypeptides from the fermentation broth, no additional formate is required to stabilize the porphyrin-containing polypeptides. In other embodiments, additional formate, or other stabilizing formulation ingredients, can be added.

A feature of the present compositions and methods is that the presence of formate in the fermentation broth increases the activity, stability, or expression levels of the porphyrin-containing polypeptide, compared to growing the same host cells in the equivalent culture medium in the absence of formate. Such increases may be determined, e.g., by measuring the amount or activity of porphyrin-containing polypeptide in cell broth, which measurement may optionally be normalized for total protein. In some embodiments, the increase is at least 5%, at least 10%, at least 15%, at least 20%, at least 25%, or even at least 30%.

The present compositions and methods are further illustrated by the following examples, which are in no way intended to limit the invention.

### EXAMPLES

### Example 1

### Production of Catalase in the Presence or Absence of Formic Acid

Pilot-scale fermentations were performed using catalase-producing host cells grown in the presence and absence of formic acid. The host cells were Trichoderma reesei cells engineered to express Aspergillis niger catalase, as described in WO2009/114380 (US2009223508), which is hereby incorporated by reference. Media conditions were substantially as described, with an initial pH of about 3.5, which was increased to about 4.25 as the initial amount of glucose was depleted and feeding was initiated in the fed-batch stage. The batch weight was about 10 kg (approximately 10 Liters (L)).

Upon the depletion of glucose in the growing culture, the cells were fed no formate (as a control), 0.62 mmol formic acid/hour/kg fermentation broth, 0.88 mmol formic acid/hour/kg fermentation broth, 0.68 mmol sodium formate/hour/kg fermentation broth, or 0.71 mmol sodium formate/hour/kg fermentation broth. The formate was fed into the culture in a regular but discontinuous manner, only because the minimum feed rate of the pump did not permit continuous feeding. The actual levels of formic acid in the fermentation broth were measured by HPLC, and varied between undetectable and 0.8 g/kg.

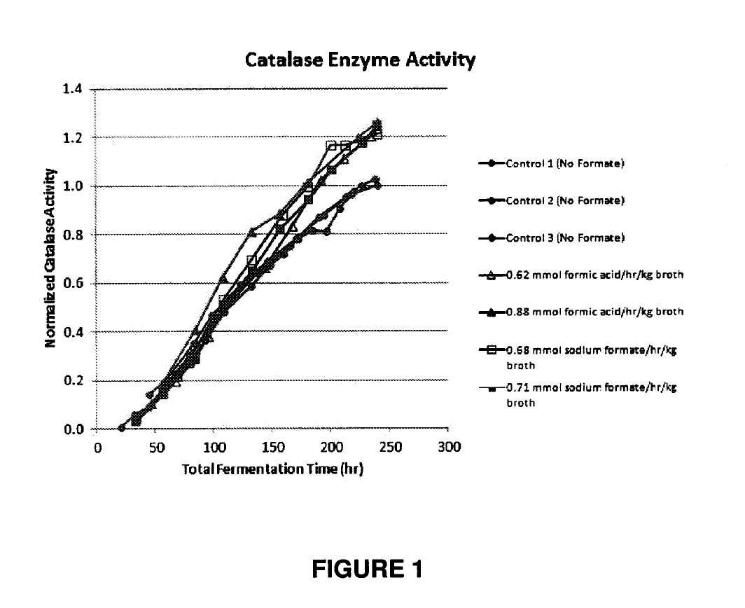

FIG. 1 shows the amount of catalase activity present in the fermentation broth (i.e., “broth activity”) over time in cell cultures grown in the presence or absence of formic acid or sodium formate. Catalase activity was measured using the Amplex Red/HRP assay (Life Technologies, Carlsbad, Calif., USA) and reported as Units catalase activity normalized by the final catalase activity value for Control 1 (no formate). Catalase activity can also be measured using the Baker catalase assay, as described in WO2009/114380 (US2009223508), or by other methods. Broth activity was consistently greater in the presence of formic acid or sodium formate.

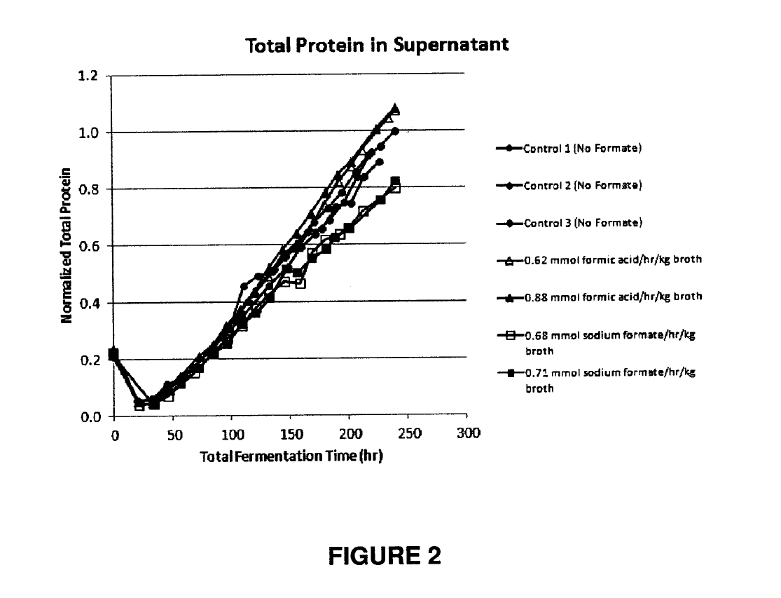

FIG. 2 shows the amount of total protein produced in cell broth supernatant over time in cell cultures grown in the presence or absence of formic acid or sodium formate. Total protein was measured by Biuret assay and values shown are normalized by the final total protein value for Control 1 (no formate). Total protein was marginally increased in the presence of formic acid and marginally decreased in the presence of sodium formate.

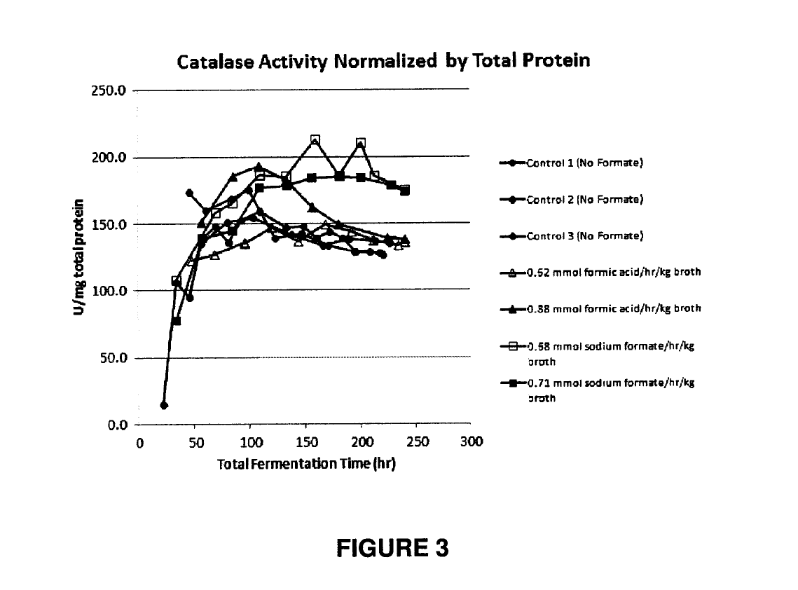

FIG. 3 shows the amount of catalase activity present in cell broth, normalized for total protein production, over time in cell cultures grown in the presence or absence of formic acid or sodium formate. Catalase activity was measured using the Amplex Red/HRP assay. Normalized broth activity was increased in the presence of formic acid or sodium formate, and particularly in the presence of sodium formate.

All references cited herein are hereby incorporated by reference in their entirety for all purposes.

1. A method for producing a porphyrin-containing polypeptide in host cells, comprising:
growing host cells that produce a porphyrin-containing polypeptide in a suitable culture medium to produce a fermentation broth;
adding to the fermentation broth an effective amount of formate; and
recovering the porphyrin-containing polypeptide produced;
wherein the presence of formate in the fermentation broth increases the activity, stability, or expression levels of the porphyrin-containing polypeptide, compared to growing the same host cells in the equivalent culture medium in the absence of formate.

2. The method of claim 1, wherein the host cells are grown to fed-batch stage prior to adding the formate.

3. The method of claim 1, wherein the growth of the host cells is substantially complete prior to adding the formate.

4. The method of claim 1, wherein the host cells are grown to a stage of being ready to induce for production of the porphyrin-containing polypeptide prior to adding the formate.

5. The method of claim 1, wherein the culture medium comprises glucose, and the glucose is substantially depleted by the host cells prior to adding the formate.

6. The method of claim 1, wherein the concentration of formate in the fermentation broth is less than or equal to about 2.0 grams formate per kilogram fermentation broth.

7. The method of claim 1, wherein the cumulative amount of formate added during fermentation is from about 0.007 to about 1.35 mole formate per kilogram (kg) fermentation broth.

8. The method of claim 1, wherein the amount of formate added is from about 0.03 to about 7.1 millimole formate per hour elapsed fermentation time per kilogram of fermentation broth.

9. The method of claim 1, wherein the amount of formate added is from about 40 to about 4400 millimole formate per hour elapsed fermentation time per kilogram dry solids of substrate.

10. The method of claim 1, wherein the amount of formate added is from about 50 to about 7500 mole formate per mole of active porphyrin-containing polypeptide.

11. The method of claim 1, wherein the formate is in the form of formic acid.

12. The method of claim 1, wherein the formate is in the form of a formate salt.

13. The method of claim 1, wherein the formate is in the form of sodium formate.

14. The method of claim 1, wherein the host cells are fungal cells.

15. The method of claim 14, wherein the host cells are filamentous fungal cells.

16. The method of claim 15, wherein the host cells are Trichoderma cells.

17. The method of claim 1, wherein the porphyrin-containing polypeptide is a heme-containing polypeptide.

18. The method of claim 1, wherein the porphyrin-containing polypeptide is a catalase enzyme.

19. A porphyrin-containing polypeptide produced by the method of claim 1.

20. The porphyrin-containing polypeptide of claim 19, wherein the polypeptide is a catalase enzyme.
